(19) United States
(12) Patent Application Publication
(10) Pub. No.: US 2008/0063619 A1
(43) Pub. Date: Mar. 13, 2008

(54) COSMETIC COMPOSITIONS WITH ETHYLENE BRASSYLATE

(75) Inventors: Amy Lynn Olsen, Monroe, CT (US); Philip Edward Miner, Newtown, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/470,635

(22) Filed: Sep. 7, 2006

(51) Int. Cl. A61K 8/73; A61K 8/89

(52) U.S. Cl. 424/70.122; 424/70.13; 424/70.31

(57) ABSTRACT

A cosmetic composition is provided which includes tapioca starch and ethylene brassylate in a cosmetically acceptable carrier. The composition provides a silky afterfeel to skin.

### BACKGROUND OF THE INVENTION

1. Field of the Invention

The invention concerns cosmetic compositions with aesthetics providing a silky afterfeel to the skin.

2. The Related Art

Proper aesthetics are essential to any successful skin cream or lotion. Without the proper feel, consumers would not buy or use even those products with the most proven dermatological benefits.

Silkiness is known to be imparted by a variety of silicone materials. Yet the silicones do not transform compositions into a powdery afterfeel. Typical disclosures of silicone materials for cosmetics are found in U.S. Pat. No. 5,972,359 (Sine et al.) and U.S. Pat. No. 6,524,598 B2 (Sunkel et al.)

Starches have been employed to enhance viscosity of liquid formulations. For instance, U.S. Pat. No. 5,824,323 (Fishman) reports skin lotion compositions with non-greasy skinfeel. These formulas can contain a variety of starches including tapioca to provide body and thickness to the lotions

U.S. Patent Application No. 2006/0034876 A1 (Cheney et al.) reports on combinations of tapioca starch and polyacrylic beads which deliver a silky rub-in aesthetic transforming into a powdery drier skin afterfeel.

Although solutions have been suggested to solve the problem of delivering silky feel to the skin, there still remains the challenge of formulating a cosmetic that in a much greater variety of formulations can achieve the aesthetically required silky smooth skin afterfeel.

### SUMMARY OF THE INVENTION

A cosmetic composition is provided which includes:
(i) from about 0.1 to about 10% by weight of tapioca starch;
(ii) from about 0.001 to about 5% by weight of ethylene brassylate;
(iii) from about 0.01 to about 10% by weight of an emulsifier;
(iv) optionally, an effective amount to provide an initial silky feel upon skin contact of a polysiloxane material;
(v) optionally, from about 0.1 to about 40% by weight of polyhydric alcohol;
(vi) optionally an effective amount to preserve of a preservative; and
(vii) a cosmetically acceptable carrier.

### DETAILED DESCRIPTION OF THE INVENTION

Now there is provided a cosmetic composition of exceptionally pleasant aesthetics. The composition delivers a silky afterfeel to skin. The afterfeel aesthetics are achieved with a combination of tapioca starch and ethylene brassylate.

Tapioca starch is a first component of the present invention. Tapioca, also known as Cassaya or Manioc is a root or tuber extract. The plant is a perennial that grows eight to twelve feet high and the roots can be as much as three feet long and five to ten inches in diameter. Cells of the tuber carry the tapioca starch. This starch is recovered by wet grinding the washed roots and continuous re-washing, resulting in a pure carbohydrate. Particularly preferred is a powder source with a median particle size ranging from about 1 to about 100 micron, (1000 nm to 100,000 nm) preferably from about 5 to about 15 micron, optimally from about 10 to about 30 micron.

The starch is commercially available under the tradename TAPIOCA PURE (28-1810) from the National Starch & Chemical Company, Division of ICI. An alternative source is TIStar Tapioca Starch sold by the Multi-Kem Corporation.

Amounts of tapioca starch may range from about 0.1 to about 10%, preferably from about 0.5 to about 5%, optimally from about 0.3 to about 1% by weight of the composition.

Advantageously the tapioca starch in certain formulations preferably is a water-insoluble partially hydrated granular substance having an average particle size ranging from about 500 to about 10,000 nm, preferably from about 1,000 to about 9,000 nm, optimally from about 3,000 to about 8,000 nm. Partially hydrated starches are achieved by heating tapioca starch in water to 50° C. and holding this temperature for from about 0.5 to about 4 hours, preferably from about 1 to about 2 hours. Partial hydration converts from about 10 to about 80% by weight of the tapioca starch to a hydrated form.

Ethylene brassylate is a second component of the present invention. This material is available from many sources including Takasago International Corporation. Amounts of ethylene brassylate may range from about 0.001 to about 5%, preferably from about 0.01 to about 1%, and more preferably from about 0.1 to about 0.5% by weight of the composition.

Advantageously the relative weight amounts of ethylene brassylate to tapioca starch may respectively range from about 1:200 to about 5:1, preferably from about 1:100 to about 3:1, more preferably from about 1:10 to about 1:1, optimally from about 1-5 to about 1:2 by weight of the composition.

Emulsifiers may also be present in cosmetic compositions of the present invention. Total concentration of the emulsifier when present may range from about 0.01 to about 10%, preferably from about 0.1 to about 5%, optimally from about 1 to about 3% by weight of the composition. The emulsifier may be selected from the group consisting of anionic, nonionic, cationic and amphoteric actives. Particularly preferred nonionic emulsifiers are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Polyhydric alcohols may be employed in certain compositions of the present invention. Typical polyhydric alcohols include glycerin (also known as glycerol), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of polyhydric alcohol when present may range from about 0.1 to about 40%, preferably from about 0.5 to about 20%, optimally from about 1 to about 10% by weight of the composition.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, methyl paraben, propyl paraben, DMDM Hydantoin, iodopropynyl butylcarbamate, 5-chloro-2-methyl-4-isothiazolin-3-one, 2-methyl-4-isothiazolin-3-one and benzyl alcohol. The preservatives should be selected having regard for the use of the composition and possible incompatibilities between the preservatives and other ingredients in the emulsion. Preservatives are preferably employed in amounts ranging from about 0.00001% to about 2% by weight of the composition.

Preferred anionic surfactants include soap, C₈-C₂₀ alkyl ether sulfates and sulfonates, C₈-C₂₀ alkyl sulfates and sulfonates, C₈-C₂₀ alkylbenzene sulfonates, C₈-C₂₀ alkyl and dialkyl sulfosuccinates, C₈-C₂₀ acyl isethionate, C₈-C₂₀ alkyl ether phosphates, C₈-C₂₀ sarcosinates, C₈-C₂₀ acyl lactylates and combinations thereof.

Polysiloxane materials may be present in compositions of this invention. The organopolysiloxane may be volatile, nonvolatile, or a mixture of volatile and non-volatile silicones. The term “nonvolatile” refers to those silicones that are liquid or solid under ambient conditions and have a flash point (under one atmosphere pressure) of at least about 100° C. The term “volatile” refers to all other silicone oils. Suitable organopolysiloxanes include polyalkylsiloxanes, cyclic polyalkylsiloxanes, and polyalkylarylsiloxanes.

Polyalkylsiloxanes can be represented by the general chemical formula R₃SiO[R₂SiO]ₓSiR₃ wherein R is an alkyl group having from one to about 30 carbon atoms (preferably R is methyl or ethyl) and x is an integer from 0 to about 10,000, chosen to achieve the desired molecular weight which can range to over about 10,000,000. Commercially available polyalkylsiloxanes include the polydimethylsiloxanes, which are also known as dimethicones. These include the Vicasil® series sold by General Electric Company and the Dow Corning® 200 series sold by Dow Corning Corporation. Dimethicones include those represented by the chemical formula (CH₃)₃SiO[(CH₃)₂SiO][CH₃RSiO]_{y}Si(CH₃)₃ wherein R is straight or branched chain alkyl having from 2 to about 30 carbon atoms and x and y are each integers of 1 or greater selected to achieve the desired molecular weight which can range to over about 10,000,000. Examples of these alkyl substituted dimethicones include cetyl dimethicone and lauryl dimethicone.

Cyclic polyalkylsiloxanes suitable for use in the composition include those represented by the chemical formula [SiR₂—O]ₙ wherein R is an alkyl group (preferably R is methyl or ethyl) and n is an integer from about 3 to about 8, more preferably from 4 to 6. Where R is methyl, these materials are typically referred to as cyclomethicones. Commercially available cyclomethicones include Dow Corning® 244 fluid which primarily contains the cyclomethicone tetramer (i.e. n=4), Dow Corning® 344 fluid which primarily contains the cyclomethicone pentamer (i.e. n=5), Dow Corning® 245 which primarily contains a mixture of the cyclomethicone tetramer and pentamer (i.e. n=4 and 5), and Dow Corning® 345 which primarily contains a mixture of the cyclomethicone tetramer, pentamer, and hexamer (i.e. n=4, 5 and 6).

Also useful are materials such as trimethylsiloxysilicate, which is a polymeric material corresponding to the general chemical formula [(CH₂)₃SiO_{1/2}]ₓ[SiO₂]_{y}, wherein x is an integer from about 1 to about 500 and y is an integer from about 1 to about 500. A commercially available trimethylsiloxysilicate is sold as a mixture with dimethicone as Dow Corning® 593 fluid.

Dimethiconols are also suitable for use in the composition. These compounds can be represented by the chemical formulas R₃SiO[R₂SiO]ₓSiR₂OH and HOR₂SiO[R₂SiO]ₓSiR₂OH wherein R is an alkyl group (preferably R is methyl or ethyl) and x is an integer from 0 to about 500, chosen to achieve the desired molecular weight. Commercially available dimethiconois are typically sold as mixtures with dimethicone or cyclomethicone (e.g. Dow Corning® 1401, 1402, 1403 and 1501 fluids). Particularly preferred is a blend with INCI name of Cyclopentasiloxane and PEG/PPG-20/15 Dimethicone commercially available from GE Silicones as SF1528.

Crosslinked organopolysiloxane elastomers may also be useful as polysiloxane materials. These may be of the emulsifying or non-emulsifying crosslinked elastomer variety. The term “non-emulsifying” defines a crosslinked organopolysiloxane elastomer from which polyoxyalkylene units are absent. The term “emulsifying” is used to mean crosslinked organopolysiloxane elastomer having at least one polyoxyalkylene unit.

Non-emulsifying silicone elastomers may be powders such as vinyl dimethicone/methicone silesquioxane crosspolymers available from Shin-Etsu as KSP-100, KSP-101, KSP-102, KSP-103, KSP-104, KSP-105, hybrid silicone powders that contain a fluoroalkyl group such as KSP-200, and hybrid silicone powders that contain a phenyl group such as KSP-300; and Dow Corning material DC 9506.

Preferred organopolysiloxane compositions are dimethicone/vinyl dimethicone crosspolymers. These are commercially available as Dow Corning (DC 9040 and DC 9045), General Electric (SFE 839), Shin Etsu (KSG-15, 16, 18 [dimethicone/phenyl vinyl dimethicone crosspolymer]), and Grant Industries (Gransii™ line of materials), and lauryl dimethicone/vinyl dimethicone crosspolymers supplied by Shin Etsu as KSG-31, KSG-32, KSG-41, KSG42, KSG43, and KSG-44.

Particularly useful emulsifying elastomers are polyoxyalkylene-modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin.

Combinations of emulsifying and non-emulsifying crosslinked siloxane elastomers may also be useful for purposes of this invention.

Amounts of the polysiloxane materials may range from about 0.1 to about 80%, preferably from about 1 to about 60%, optimally from about 5 to about 40% by weight of the composition.

Fatty acids having from 10 to 30 carbon atoms may in certain formulations also be suitable for compositions of the present invention. Illustrative of this category are pelargonic, lauric, myristic, palmitic, stearic, isostearic, hydroxystearic, oleic, linoleic, ricinoleic, arachidic, behenic and erucic acids. Particularly preferred is stearic acid. Amounts of the fatty acid may range from about 0.1 to about 20%, preferably from about 0.5 to about 10%, optimally from about 1 to about 5% by weight of the composition.

Another component which may be utilized in compositions of the present invention is that of polyacrylic beads. Especially useful are polymethyl methacrylate, polyethyl methacrylate, polyethyl acrylate and polymethyl acrylate polymers. Most preferred are polymethyl methacrylate beads. The beads can range in number average particle size from about 1 to about 50 micron, preferably from about 3 to about micron, optimally from about 5 to about 10 micron. These beads may be crosslinked or non-crosslinked, but crosslinking is preferred. The beads may have an Oil Absorbance ranging from about 60 to about 300 ml/100 g, preferably from about 70 to about 180 ml/100 g, as defined by ASTM-D281-31. Beads of polymethyl methacrylate are sold under the trademark Ganzpearl, available from Presperse Inc., Piscataway, N.J. 08854. Most preferred is Ganzpearl® GMP-0820 with number average particle size of about 8 micron and an Oil Absorbance of about 170 ml/100 g.

Amounts of the polyacrylate beads may range from about 0.001 to about 5%, preferably from about 0.01 to about 1%, optimally from about 0.1 to about 0.5% by weight of the composition.

Compositions of this invention will include a cosmetically acceptable carrier. Amounts of the carrier may range from 1 to 99.9%, preferably from about 50 to about 95%, optimally from about 80 to about 90%. Among the useful carriers are water, emollients, fatty alcohols, thickeners and combinations thereof. The carrier may be aqueous, anhydrous or an emulsion. Preferably the compositions are aqueous, especially water and oil emulsions of the W/O or O/W variety. Water when present may be in amounts ranging from about 1 to about 95%, preferably from about 20 to about 70%, optimally from about 35 to about 60% by weight.

Emollient materials may serve as cosmetically acceptable carriers. These may be in the form of natural or synthetic esters and hydrocarbons. Amounts of the emollients may range anywhere from about 0.1 to about 95%, preferably between about 1 and about 50% by weight.

Among the ester emollients are:

a) Alkenyl or alkyl esters of fatty acids having 10 to 20 carbon atoms. Examples thereof include isoarachidyl neopentanoate, isononyl isonanonoate, oleyl myristate, isopropyl myristate, oleyl stearate, and oleyl oleate.

b) Ether-esters such as fatty acid esters of ethoxylated fatty alcohols.

c) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

d) Wax esters such as beeswax, spermaceti wax and tribehenin wax.

e) Sterols esters, of which cholesterol fatty acid esters are examples thereof.

f) Sugar esters of fatty acids such as sucrose polybehenate and sucrose polycottonseedate.

g) Natural esters useful in this invention are sunflower seed oil, safflower oil, cottonseed oil, olive oil, jojoba and mixtures thereof.

Hydrocarbons which are suitable cosmetically acceptable carriers include petrolatum, mineral oil, C₁₁-C₁₃ isoparaffins, polyalphaolefins, and especially isohexadecane, available commercially as Permethyl 101A from Presperse Inc.

Fatty alcohols having from 10 to 30 carbon atoms are another useful category of cosmetically acceptable carrier. Illustrative are stearyl alcohol, lauryl alcohol, myristyl alcohol and cetyl alcohol. Amounts may range from about 0.05 to about 20%, preferably from about 0.1 to about 2% by weight of the composition.

Thickeners can be utilized as part of the cosmetically acceptable carrier of compositions according to the present invention. Typical thickeners include polyacrylamides (e.g. Sepigel 305®), acryloyidimethyltaurate polymers and copolymers (e.g. Aristoflex AVC), crosslinked acrylates (e.g. Carbopol 982®), hydrophobically-modified acrylates (e.g. Carbopol 1382®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenum, pectin and combinations of these gums. Inorganics may also be utilized as thickeners, particularly clays such as bentonites and hectorites, fumed silicas, and silicates such as magnesium aluminum silicate (Veegum®). Amounts of the thickener may range from about 0.0001 to about 10%, usually from about 0.001 to about 1%, optimally from about 0.01 to about 0.5% by weight of the composition.

Cosmetic compositions of the present invention may be in any form. These forms may include lotions, creams, roll-on formulations, sticks, masks, mousses, aerosol and non-aerosol sprays and pad-applied (e.g. wipe) formulations.

Sunscreen actives may also be included in compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide and zinc oxide. Amounts of the sunscreen agents when present may generally range from 0.1 to 30%, preferably from 2 to 20%, optimally from 4 to 10% by weight.

Compositions of the present invention may also contain vitamins and their derivatives. By the term “derivatives” is meant C₁-C₄₀ alkyl esters, salts or isomers. The vitamins may include Vitamin A and derivatives (e.g. retinyl palmitate, retinyl linoleate, retinyl acetate, retinoic acid), Vitamin B (including Niacinamide, DL-Panthenol, Pyridoxine Palmitate, Folic Acid, Carotin, Biotin), Vitamin C (including magnesium ascorbyl phosphate, ascorbyl tetraisopalmitate, ascorbyl glucoside), Vitamin D, Vitamin E (including tocopherol palmitate, tocopherol acetate and the alpha-, beta- and gamma-isomers of tocopherol) and combinations thereof. Total amount of vitamins when present in compositions according to the present invention may range from 0.00001 to 10%, preferably from 0.01% to 1%, optimally from 0.1 to 0.5% by weight of the composition.

Skin lightening agents may be included in the compositions of the invention. Illustrative substances are placental extract, lactic acid, niacinamide₁ arbutin, kojic acid, resorcinol and derivatives including 4-substituted resorcinois and combinations thereof. Amounts of these agents may range from about 0.1 to about 10%, preferably from about 0.5 to about 2% by weight of the composition.

Desquamation agents are further optional components. Illustrative are the alpha-hydroxycarboxylic acids and beta-hydroxycarboxylic acids, Among the former are salts of glycolic acid, lactic acid and malic acid, Salicylic acid is representative of the beta-hydroxycarboxylic acids. Amounts of these materials when present may range from about 0.01 to about 15% by weight of the composition.

A variety of herbal extracts may optionally be included in compositions of this invention. Illustrative are green tea, chamomile, licorice, lavender, grape seed and extract combinations thereof. The extracts may either be water soluble or water-insoluble carried in a solvent which respectively is hydrophilic or hydrophobic. Water and ethanol are the preferred extract solvents.

Flavonoids may also be included in compositions of this invention. The flavonoids may be selected from isoflavonoids, flavonols and combinations thereof. Particularly useful are glucosyl hesperidin and rutin. Amounts of these agents may range from about 0.000001 to about 5% by weight of the composition.

Anti-microbial agents may also be included in the compositions of this invention. Illustrative are trichlosan, trichlocarban, Octopyrox® and zinc pyrithione. Amounts may range from about 0.01 to about 5%, preferably from about 0.1 to about 0.5% by weight of the composition.

Colorants, fragrances, opacifiers and abrasives may also be included in compositions of the present invention. Each of these substances may range from about 0.05 to about 5%, preferably between 0.1 and 3% by weight.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

It should be noted that in specifying any range of concentration or amount, any particular upper concentration can be associated with any particular lower concentration or amount.

The following examples will more fully illustrate the embodiments of this invention. All parts, percentages and proportions referred to herein and in the appended claims are by weight unless otherwise illustrated.

### EXAMPLE 1

Typical body lotions according to the present invention are illustrated in the Examples of Table I.

TABLE I

| | | Example (Weight %) | | | | | | | |
|---|---|---|---|---|---|---|---|---|---|
| Trademark | Chemical Name | 1 | 2 | 3 | 4 | 5 | 6 | 7 | 8 |
| Pristerene 4911 | Stearic Acid | 6.5 | 7.5 | 8.5 | 1.0 | 2.0 | 3.0 | 4.0 | 5.0 |
| Ritasynt | Glycerol Monostearate/ | 1.5 | 1.5 | 1.5 | 1.5 | 1.5 | 1.5 | 1.5 | 1.5 |
| | Stearamide AMP | | | | | | | | |
| Kessco GMS | Glycerol Monostearate | 0.5 | 1.0 | 0.5 | 0.5 | 1.0 | 0.5 | 1.5 | 1.5 |
| Cetyl Alcohol | Cetyl Alcohol | 0.5 | 0.5 | 0.5 | 0.5 | 0.5 | 0.5 | 0.5 | 0.5 |
| IPM | Isopropyl Myristate | 7.5 | 5.0 | 5.0 | 5.0 | 5.0 | 7.5 | 10.0 | 5.0 |
| Silicone Fluid 50 cts | Dimethicone | 4.0 | 5.0 | 6.0 | 2.0 | 2.0 | 4.0 | 4.0 | 8.0 |
| Disodium EDTA | Disodium EDTA | 0.05 | 0.05 | 0.05 | 0.05 | 0.05 | 0.05 | 0.05 | 0.05 |
| Glycerin | Glycerin | 5.0 | 5.0 | 5.0 | 5.0 | 5.0 | 5.0 | 10.0 | 10.0 |
| Veegum | Magnesium Aluminum | 0.3 | 0.3 | 0.3 | 0.3 | 0.3 | 0.3 | 0.3 | 0.3 |
| | Silicate | | | | | | | | |
| Keltrol CG 100 | Xanthan Gum | 0.1 | 0.1 | 0.1 | 0.1 | 0.1 | 0.1 | 0.1 | 0.1 |
| Carbopol 934 (2% | Carbomer | 9.0 | 9.0 | 9.0 | 12.0 | 12.0 | 6.5 | 6.5 | 6.5 |
| Active) | | | | | | | | | |
| Titanium Dioxide | Titanium Dioxide | 0.1 | 0.1 | 0.1 | 0.1 | 0.1 | 0.1 | 0.1 | 0.1 |
| Triethanolamine | Triethanolamine | 1.0 | 1.0 | 1.0 | 1.0 | 1.0 | 1.0 | 1.0 | 1.0 |
| (99%) | | | | | | | | | |
| DC 245 Fluid | Cyclopentasiloxane | 2.0 | 2.0 | 2.0 | 2.0 | 2.0 | 2.0 | 2.0 | 2.0 |
| DC 1501 | Dimethiconol | 0.5 | 0.5 | 0.5 | 0.5 | 0.5 | 0.5 | 0.5 | 0.5 |
| Ganzpearl GMP | Methyl Methacrylate | 0.25 | 0.25 | 0.5 | 0.5 | 0.5 | 0.75 | 0.75 | 0.25 |
| 0820 | Crosspolymer | | | | | | | | |
| GE SF 1528 | Dimethicone | 0.75 | 1.0 | 0.75 | 0.75 | 2.0 | 2.0 | 5.0 | 0.25 |
| | Copolyol/Cyclomethicone | | | | | | | | |
| Tapioca Pure | Tapioca Starch | 1.75 | 1.75 | 2.0 | 2.0 | 5.0 | 5.0 | 2.25 | 1.75 |
| Glydant Plus | Iodopropynyl | 0.09 | 0.09 | 0.09 | 0.09 | 0.09 | 0.09 | 0.09 | 0.09 |
| | Butylcarbamate/DMDM | | | | | | | | |
| | Hydantoin | | | | | | | | |
| Ethylene Brassylate | Ethylene Brassylate | 0.1 | 0.5 | 1.0 | 2.0 | 1.0 | 1.5 | 0.8 | 0.3 |
| Fragrance | Fragrance | 0.15 | 0 | 1.0 | 0.75 | 0.75 | 0.15 | 0.15 | 1.1 |
| | Water | Balance | Balance | Balance | Balance | Balance | Balance | Balance | Balance |

The formulations of Table I are blended in the following manner. Above specified quantities of water, disodium EDTA, titanium dioxide and gum xanthan/glycerin slurry (2%) are charged to a batching vessel. These components are mixed for 5 minutes and then the Carbomer is added as a solution. Heat is applied and the mixed contents are held at 80° C. for 15 minutes. The remaining glycerin is added. The oil phase ingredients are then charged to a separate vessel. Light mixing is begun and heat applied to achieve 80° C. Slowly, the oil phase is added to the water phase under moderate mixing while maintaining temperature. All remaining water is charged to the vessel. Then the contents are homogenized at 80° C. for 1 minutes. Contents are then cooled to 38-40° C. accompanied by large sweep (75 rpm) mixing. Glydant Plus is added at 55° C. Cooling is continued accompanied by slow addition at no higher than 50° C. of DC 1501, fragrance, ethylene brassylate and tapioca along with the various minor ingredients.

### EXAMPLE 9

Illustrated herein is a skin cream according to the present invention.

TABLE II

| INGREDIENT | WEIGHT % |
|---|---|
| Glycerin | 6.95 |
| Niacinamide | 5.00 |
| Tapioca Starch | 5.00 |
| Permethyl 101A¹ | 3.00 |
| Sepigel 305² | 2.50 |
| DC-1403³ | 2.00 |
| Isopropyl Isostearate | 1.50 |
| Arlatone 2121⁴ | 1.00 |
| Cetyl Alcohol | 0.75 |
| SEFA Cottonate⁵ | 0.70 |
| Tocopherol Acetate | 0.50 |
| Panthenol | 0.50 |
| Stearyl Alcohol | 0.50 |
| Titanium Dioxide | 0.40 |
| Ethylene Brassylate | 0.35 |
| Disodium EDTA | 0.10 |
| Glydant Plus⁶ | 0.10 |
| PEG-100 Stearate | 0.10 |
| Stearic Acid | 0.10 |
| Purified Water | Balance |

¹Isohexadecane, Presperse Inc., South Plainfield, NJ
²Polyacrylamide(and)C13–14 Isoparaffin(and) Laureth-7, Seppic Corporation, Fairfield, NJ
³dimethicone(and)dimethiconol, Dow Corning Corp. Midland, MI
⁴Sorbitan Monostearate and Sucrococoate, ICI Americas Inc., Wilmington, DE
⁵Sucrose ester of fatty acid
⁶DMDM Hydantoin (and) Iodopropynyl Butylcarbamate, Lonza Inc., Fairlawn, NJ

### EXAMPLE 10

Another oil-in-water emulsion according to the present invention is described under Table III.

TABLE III

| INGREDIENT | WEIGHT % |
|---|---|
| Phase A | |
| Distilled Water | balance |
| Phase B | |
| Glycerin | 5.00 |
| Titanium Dioxide | 0.75 |
| Phase C | |
| Glycerin | 1.00 |
| Disodium EDTA | 0.10 |
| Carbopol ® 954 | 0.68 |
| Carbopol ® 1382 | 0.10 |
| Tapioca Starch | 2.50 |
| Phase D | |
| Cetyl Alcohol | 0.70 |
| Stearyl Alcohol | 0.50 |
| Stearic Acid | 0.10 |
| PEG-100 Stearate | 0.10 |
| Glycerol Monostearate | 1.00 |
| Dimethicone | 2.00 |
| Cyclomethicone | 0.65 |
| Tocopherol Acetate | 0.10 |
| Niacinamide | 2.00 |
| Phase E | |
| Distilled Water | 2.00 |
| NaOH | To neutralize |
| Phase F | |
| Urea | 2.00 |
| D-Panthenol | 0.10 |
| Distilled Water | 5.00 |
| Phase G | |
| Glydant Plus | 0.10 |
| Glycerin | 1.00 |
| Distilled Water | 1.00 |
| Phase H | |
| Ethylene Brassylate | 1.00 |
| Methyl Isostearate | 1.50 |
| Ganzpearl ® GMP 0820 | 0.50 |
| Retinol | 0.50 |
| Butylated Hydroxy Toluene | 0.05 |
| Tween ® 20 | 1.00 |

### EXAMPLE 11

A hand cream according to the present invention is herein reported under Table IV.

TABLE IV

| Ingredient | Weight % |
|---|---|
| Distearyldimonium chloride (Varisoft TA100 ®) | 4.1 |
| Cetyl Alcohol | 2.9 |
| Steareth 21 | 1.7 |
| Stearyl Stearate | 0.01 |
| Petrolatum | 0.3 |
| Dimethicone 200/200 | 1.0 |
| Mineral Oil 1000SUS | 2.8 |
| Isopropyl palmitate | 4.0 |
| Soy Sterol | 0.1 |
| Stearic Acid | 0.1 |
| Lecithin | 0.1 |
| Borage Seed Oil | 0.1 |
| Vitamin E Acetate | 0.1 |
| Propylparaben | 0.1 |
| Sodium Chloride | 0.2 |
| Glycerin | 10.0 |
| Titanium Dioxide | 0.1 |
| Methylparaben | 0.2 |
| Tapioca Starch | 0.5 |
| DMDM Hydantoin | 0.3 |
| Ethylene Brassylate | 0.2 |
| Water | Balance |

### EXAMPLE 12

Herein is reported a conditioning body butter with the formula outlined under Table V.

TABLE V

| | WEIGHT |
|---|---|
| INGREDIENT | % |
| Stearic Acid | 2.5 |
| Glycol Stearate and Stearamide AMP | 1.5 |
| Glyceryl Monostearate | 0.7 |
| Cetyl alcohol | 0.4 |
| Dimethicone | 2.5 |
| Petrolatum | 0.5 |
| Ethylhexylcocoate & Hydrogenated didecene | 2.5 |
| Cocoa Butter | 1.3 |
| Microcrystalline Wax | 0.3 |
| Paraffin Wax | 0.3 |
| Propylparaben | 0.1 |
| Disodium EDTA | 0.1 |
| Methylparaben | 0.2 |
| PEG-90 Diisostearate (Hydramal PGDS) | 3.0 |
| Viscolam AP64 (Sodium Acrylate/Sodium Acryloyldimethyl | 1.5 |
| Taurate Copolymer, Hydrogenated Polydecene, Laureth-8) | |
| Triethanolamine | 0.8 |
| Xanthan Gum | 0.1 |
| Glycerin | 15.0 |
| Cyclopentasiloxane (DC 245) | 4.0 |
| Tapioca | 2.0 |
| Cyclopentasiloxane and Dimethiconol (DC1501) | 0.5 |
| Cyclopentasiloxane and Dimethicone Copolyol (SF 1528) | 0.5 |
| Phenoxyethanol | 0.4 |
| Ethylene Brassylate | 0.2 |
| Water | Balance |

### EXAMPLE 13

Herein is reported a healing foot cream having a formula outlined under Table VI.

TABLE VI

| INGREDIENT | WEIGHT % |
|---|---|
| Disodium EDTA | 0.1 |
| Magnesium Aluminum Silicate (Veegum | 0.3 |
| Ultra) | |
| Glycerin | 3.0 |
| Butylene Glycol | 2.0 |
| Methylparaben | 0.2 |
| Xanthan Gum | 0.2 |
| Hydroxyethylcellulose (Natrosol) | 0.1 |
| Stearic Acid | 3.6 |
| Glycol Stearate and Stearamide AMP | 2.1 |
| Glyceryl Stearate | 1.0 |
| Cetyl Alcohol | 0.6 |
| PEG-100 Stearate | 1.8 |
| Petrolatum | 12.0 |
| Caprylic/Capric Triglyceride | 1.8 |
| Dimethicone (200 cts) | 1.0 |
| Ethylhexyl Methoxycinnamate | 0.5 |
| Sunflower Seed oil | 1.0 |
| Propylparaben | 0.1 |
| Glydant (DMDM Hydantoin) | 0.3 |
| Lactic Acid and Potassium Lactate | 8.0 |
| Ethylene Brassylate | 0.6 |
| Tapioca Starch | 3.5 |
| Deionized Water | Balance |

### EXAMPLE 14

Herein is reported a moisture locking lotion with the formula as recorded in Table VII. In this formula the tapioca starch is added last to the oil and water phases at a temperature no higher than 40° C. This ensures that the tapioca starch becomes only partially hydrated.

TABLE VII

| INGREDIENT | WEIGHT % |
|---|---|
| Oil Phase | |
| Stearic Acid | 4.5 |
| Glycol Stearate and Stearamide AMP | 1.0 |
| Glyceryl Monostearate | 0.7 |
| Cetyl alcohol | 0.4 |
| Petrolatum | 6.5 |
| Dimethicone (200 cts) | 1.0 |
| Isopropyl Isostearate | 1.0 |
| Propylparaben | 0.1 |
| Water Phase | |
| DI Water | Balance |
| Disodium EDTA | 0.1 |
| Methylparaben | 0.2 |
| Glycerin USP | 12.0 |
| Titanium Dioxide (Water dispersible) | 0.1 |
| Magnesium Aluminum Silicate | 0.2 |
| (Veegum) | |
| Carbopol 934 2% solution | 9.0 |
| Triethanolamine | 1.1 |
| Cool Down Phase | |
| DI Water | 10.0 |
| Tapioca Starch | 1.0 |
| Phenoxyethanol | 0.4 |
| Ethylene Brassylate | 0.1 |

### EXAMPLE 15

A series of experiments were conducted to evaluate skinfeel performance through instrumentation. Various combinations of tapioca starch and ethylene brassylate were evaluated for slip friction as a function of time. This procedure measures friction during the first 10 minutes of drying after application. The test is conducted in an environmentally controlled chamber at 21° C. and 20% relative humidity. Sample size of 100 microliters is spread on a Lucite table over a 6 by 5 inch area (2.54 cm to the inch). The table is attached to the cross-head of an Instron Model 4501 Materials Testing System. A 3 by 1 inch aluminum sled covered with a 100% rayon nonwoven is pulled across the same area at a rate of 10 cm/min, starting one minute after application and repeated each minute for ten minutes. The integral of force vs. a distance of 50 mm (i.e.—amount of work, units of gram-mm) is calculated for each of the intervals. Three runs are made of each product.

Table VIII identifies the amounts of tapioca and ethylene brassylate for each sample in a base formula similar to that of Example 1. Slip friction results are reported in Table IX.

TABLE VIII

| | | Ethylene Brassylate | Corn Starch |
|---|---|---|---|
| Sample No. | Tapioca (weight %) | (weight %) | (weight %) |
| A | 0 | 2.0 | 0 |
| B | 0.5 | 1.5 | 0 |
| C | 2.0 | 0 | 0 |
| D | 1.0 | 1.0 | 0 |
| E | 1.5 | 0.5 | 0 |
| F | 0 | 1.0 | 1.0 |

TABLE IX

| Sample No. | Slip Work Load (gram-mm) |
|---|---|
| A | 768.2 |
| B | 743.8 |
| C | 742.3 |
| D | 662.9 |
| E | 633.1 |
| F | 728.5 |

The results shown in Table IX demonstrate that a combination of ethylene brassylate and tapioca starch (Samples D and E) provide better slip results (lower friction) than either of the separate materials (Samples A and C). Further, the results of ethylene brassylate with tapioca are much better than the ethylene brassylate combination with corn starch. Compare Sample C to Sample F.

While particular embodiments of the subject invention have been described it will be obvious to those skilled in the art that various changes and modifications to the subject invention can be made without departing from the spirit and scope of the invention. It is intended to cover, in the appended claims, all such modifications that are within the scope of the subject invention.

What is claimed is:

1. A cosmetic composition comprising:
(i) from about 0.1 to about 10% by weight of tapioca starch;
(ii) from about 0.001 to about 5% by weight of ethylene brassylate;
(iii) from about 0.01 to about 10% by weight of an emulsifier;
(iv) optionally, an effective amount to provide an initial silky feel upon skin contact of a polysiloxane material;
(v) optionally, from about 0.1 to about 40% by weight of polyhydric alcohol;
(vi) optionally, an effective amount to preserve of a preservative; and
(vii) a cosmetically acceptable carrier.

2. The cosmetic composition according to claim 1 wherein the tapioca starch is present in an amount from about 0.5 to about 5% by weight of the composition.

3. The cosmetic composition according to claim 1 wherein ethylene brassylate is present in an amount from about 0.1 to about 0.5% by weight of the composition.

4. The composition according to claim 1 wherein ethylene brassylate and tapioca starch are present in a relative amount ranging from about 1:200 to about 5:1.

5. The cosmetic composition according to claim 1 wherein ethylene brassylate and tapioca starch are present in a relative weight ratio from about 1:10 to about 1:1.

6. The cosmetic composition according to claim 1 wherein the tapioca starch is a partially hydrolyzed material.

7. The cosmetic composition according to claim 1 wherein the polyhydric alcohol is glycerin.

8. The cosmetic composition according to claim 1 wherein the polysiloxane material is present from about 0.1 to about 80% by weight of the composition.
